(19) United States
(12) Patent Application Publication
(10) Pub. No.: US 2025/0352446 A1
(43) Pub. Date: Nov. 20, 2025

(54) COMPOSITIONS FOR PREVENTING, REDUCING, OR AMELIORATING MALODOROUS ALDEHYDES AND KETONES, AND USES THEREOF

(71) Applicant: Firmenich SA, Satigny (CH)

(72) Inventors: Arnaud STRUILLOU, Satigny (CH); Glenn Paul Johannes VERHOVNIK, Satigny (CH); Pauline LEDERREY, Satigny (CH)

(73) Assignee: Firmenich SA, Satigny (CH)

(21) Appl. No.: 18/872,808

(22) PCT Filed: Jun. 15, 2023
(86) PCT No.: PCT/EP2023/066036

§ 371 Date: Dec. 7, 2024

(30) Foreign Application Priority Data
Jun. 21, 2022 (EP) 22180210.1

(51) Int. Cl. A61K 8/37; A61K 8/41; A61Q 13/00; A61Q 15/00

(52) U.S. Cl. CPC A61K 8/37; A61K 8/41; A61Q 13/00; A61Q 15/00

(57) ABSTRACT

The present disclosure relates to compositions, typically fragrance compositions, that actively reduce malodorous aldehydes and ketones. The present disclosure also relates to uses and methods of using such compositions for preventing, reducing, or ameliorating malodor caused by such malodorous aldehydes and ketones.

### CROSS-REFERENCE TO RELATED APPLICATIONS

The present application claims the benefit of priority of European Application No. 22180210.1, filed Jun. 21, 2022, the entire content of which is explicitly incorporated herein by this reference.

### FIELD OF THE DISCLOSURE

The present disclosure relates to compositions, typically fragrance compositions, that actively reduce malodorous aldehydes and ketones. The present disclosure also relates to uses and methods of using such compositions for preventing or reducing malodor caused by such malodorous aldehydes and ketones.

### BACKGROUND OF THE DISCLOSURE

Body odor is an umbrella term for natural smells originating from a person. The human body can produce a range of substances that often degrade or are transformed by natural processes, such as, for example, autoxidation, bacterial action, enzymatic action, and the like. The degradation or transformation of naturally occurring compounds secreted on the skin often result in compounds that have an unpleasant smell. In small amounts, such smells may not be noticeable. However, an excessive accumulation of such degradation or transformation products on the skin may lead to strong unpleasant smells.

For instance, in some areas in India, many people treat their hair with hair oils and only wash their hair about once a week. Due to this routine, some malodorous aldehydes and ketones are created from the oxidation of scalp sebum and/or fatty acids from the hair oil.

In addition, so-called “old person smell” is a characteristic odor that occurs in middle aged and elderly people as the result of degradation of lipid hydroperoxides in their skin, releasing malodorous aldehydes, primarily the compound 2-nonenal (see S. Haze et al, J. Invest. Dermatol. 116:520-524 2001), and perhaps other lipid oxidation products.

Body odor not only arises from a person's skin, hair, or scalp, but also from the person's clothing as well. While an article of clothing is being worn, sweat and oils from the skin of the wearer can be transferred to and absorbed by an article of clothing. By autoxidation, bacterial action, or enzymatic action, the skin lipids which become deposited onto the cloth can be oxidized to intermediate hydroperoxides that further decompose to release malodorous aldehydes and other compounds. This can also lead to body odor that can be perceived by the wearer and/or persons near the wearer.

Thus, there is an ongoing need for compositions and methods for preventing, reducing, or ameliorating the olfactive perception of malodor resulting from the accumulation of malodorous aldehydes and ketones on a person or other surfaces.

### SUMMARY OF THE DISCLOSURE

The following aspects of the present disclosure seek to address one or more of the problems described hereinabove.

In a first aspect, the present disclosure relates to a fragrance composition, comprising:
a) an active component comprising at least one 1,3-dicarbonyl compound and, optionally, at least one basic compound, wherein the amount of the 1,3-dicarbonyl compound is at least 10%, typically at least 20%, more typically at least 30%, by weight relative to the total weight of the composition; and
b) at least one ingredient selected from the group consisting of a perfumery carrier and a perfumery base, wherein the at least one ingredient is different from the at least one 1,3-dicarbonyl compound.

In a second aspect, the present disclosure relates to a perfumed consumer product comprising the fragrance composition described herein.

In a third aspect, the present disclosure relates to use of the fragrance composition or perfumed consumer product described herein to prevent, reduce, or ameliorate malodorous aldehydes and ketones from the hair, scalp, skin, or any combination thereof, of a subject.

In a fourth aspect, the present disclosure relates to use of the fragrance composition or perfumed consumer product described herein to prevent, reduce, or ameliorate malodorous aldehydes and ketones from any inanimate surface, typically a fabric.

### DETAILED DESCRIPTION

As used herein, the terms “a”, “an”, or “the” means “one or more” or “at least one” unless otherwise stated.

While compositions and methods are described in terms of “comprising,” “containing,” or “including” various components or steps, the compositions and methods can also “consist essentially of” or “consist of” the various components, substances and steps. As used herein the term “consisting essentially of” shall be construed to mean including the listed components, substances or steps and such additional components, substances or steps which do not materially affect the basic and novel properties of the composition or method. In some embodiments, a composition in accordance with embodiments of the present disclosure that “consists essentially of” the recited components or substances does not include any additional components or substances that alter the basic and novel properties of the composition.

Unless defined otherwise, all technical and scientific terms used herein have the same meaning as is commonly understood by one of skill in the art to which this specification pertains.

It should be understood that any numerical range recited herein is intended to include all sub-ranges subsumed therein. For example, a range of “1 to 10” is intended to include all sub-ranges between and including the recited minimum value of 1 and the recited maximum value of 10; that is, having a minimum value equal to or greater than 1 and a maximum value of equal to or less than 10. Because the disclosed numerical ranges are continuous, they include every value between the minimum and maximum values. Unless expressly indicated otherwise, the various numerical ranges specified in this application are approximations.

As used herein, and unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

Throughout the present disclosure, various publications may be incorporated by reference. Should the meaning of any language in such publications incorporated by reference conflict with the meaning of the language of the present disclosure, the meaning of the language of the present disclosure shall take precedence, unless otherwise indicated.

In the first aspect, the present disclosure relates to a fragrance composition, comprising:
a) an active component comprising at least one 1,3-dicarbonyl compound and, optionally, at least one basic compound, wherein the amount of the 1,3-dicarbonyl compound is at least 10%, typically at least 20%, more typically at least 30%, by weight relative to the total weight of the composition; and
b) at least one ingredient selected from the group consisting of a perfumery carrier and a perfumery base, wherein the at least one ingredient is different from the at least one 1,3-dicarbonyl compound.

Without wishing to be bound to any particular theory, the fragrance composition described herein makes use of the Knoevenagel condensation, in which the at least one 1,3-dicarbonyl compound may form a carbanion that undergoes a nucleophilic addition to the carbonyl carbon of malodorous aldehydes or ketones. Followed by the elimination of H₂O, an unsaturated condensation product is formed, which is no longer malodorous or only slightly malodorous.

The 1,3-dicarbonyl compound is not particularly limited. However, in an embodiment, the at least one 1,3-dicarbonyl compound is a compound represented by formula (I),

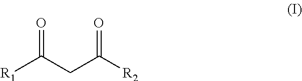

wherein
R₁ and R₂ are each, independently, H, OH, C₁-C₂₀ alkyl, typically C₁-C₁₀ alkyl, more typically, C₁-C₆ alkyl, or C₁-C₂₀ alkoxy, typically C₁-C₁₀ alkoxy, more typically C₁-C₆ alkoxy; or
R₁ and R₂ together form a 5- or 6-membered carbocyclic ring, optionally interrupted by one or more heteroatoms; wherein R₁ and R₂ may optionally comprise one or more substituents selected from the group consisting of halogens, aryl, alkoxy, CN, NO₂, OH, SH, SCH₃, NH₂, COOH, and CONH₂.

As used herein, the terminology “Cx-Cy” in reference to an organic group, wherein x and y are each integers, means that the group may contain from x carbon atoms to y carbon atoms per group.

As used herein, the term “alkyl” means a monovalent straight or branched saturated hydrocarbon radical, more typically, a monovalent straight or branched saturated (C₁-C₄₀) hydrocarbon radical, such as, for example, methyl, ethyl, n-propyl, isopropyl, n-butyl, isobutyl, tert-butyl, hexyl, 2-ethylhexyl, octyl, decyl, lauryl, hexadecyl, octadecyl, eicosyl, behenyl, tricontyl, and tetracontyl.

As used herein, the term “alkoxy” means a monovalent radical denoted as —O-alkyl, wherein the alkyl group is as defined herein. Examples of alkoxy groups, include, but are not limited to, methoxy, ethoxy, n-propoxy, isopropoxy, n-butoxy, isobutoxy, and tert-butoxy.

As used herein, the term “aryl” means a monovalent unsaturated hydrocarbon radical containing one or more six-membered carbon rings in which the unsaturation may be represented by three conjugated double bonds. Aryl radicals include monocyclic aryl and polycyclic aryl. Polycyclic aryl refers to a monovalent unsaturated hydrocarbon radical containing more than one six-membered carbon ring in which the unsaturation may be represented by three conjugated double bonds wherein adjacent rings may be linked to each other by one or more bonds or divalent bridging groups or may be fused together. Examples of aryl radicals include, but are not limited to, phenyl, anthracenyl, naphthyl, phenanthrenyl, fluorenyl, and pyrenyl.

Any substituent or radical described herein may optionally be substituted at one or more carbon atoms with one or more, same or different, substituents described herein. For instance, an alkyl group may be further substituted with an aryl group or another alkyl group. Any substituent or radical described herein may also optionally be substituted at one or more carbon atoms with one or more substituents selected from the group consisting of halogen, such as, for example, F, Cl, Br, and I; nitro (NO₂), cyano (CN), hydroxy (OH), thio (SH), methylthio (SCH₃), amino (NH₂), carboxyl (COOH), amido (CONH), and the like.

Exemplary 1,3-dicarbonyl compounds include, but are not limited to, malonic acid, malonic acid esters, acetoacetic acid, acetoacetic acid esters, Meldrum's acid, and the like.

In an embodiment, R₁ and R₂ are each, independently, C₁-C₆ alkyl or C₁-C₆ alkoxy.

In another embodiment, R₁ is C₁-C₆ alkyl, typically ethyl, and R₂ is C₁-C₆ alkoxy, typically ethoxy.

In yet another embodiment, the at least one 1,3-dicarbonyl compound is ethyl acetoacetate.

Generally, the amount of the 1,3-dicarbonyl compound in the fragrance composition is at least 10%, typically at least 20%, more typically at least 30%, by weight relative to the total weight of the composition.

In an embodiment, the amount of the at least one 1,3-dicarbonyl compound is from 10% to 99%, typically 20% to 99%, more typically 30% to 99%, still more typically 50% to 99%, by weight relative to the total weight of the composition.

The fragrance composition may optionally comprise at least one basic compound. Without wishing to be bound by theory, the at least one basic compound activates an acidic hydrogen atom on the at least one 1,3-dicarbonyl compound to facilitate forming the carbanion that undergoes a nucleophilic addition to the carbonyl carbon of malodorous aldehydes or ketones. While some malodor reduction still takes place in the absence of the basic compound, it is less efficient. Thus, in an embodiment, the fragrance composition comprises both the at least one 1,3-dicarbonyl compound and the at least one basic compound.

The basic compound may be any compound capable of deprotonating the acidic proton between the two carbonyl groups of the 1,3-dicarbonyl compound, thus forming a stabilized carbanion which reacts with malodorous aldehydes and/or ketones. Suitable basic compounds include, but are not limited to, alkali metal and alkaline earth metal salts of hydroxide, carbonate, bicarbonate, phosphate, dihydrogenphosphate, monohydrogenphosphate, sulfate, hydrogensulfate; and amines.

The amount of the at least one basic compound is not particularly limited. In an embodiment, the amount of the basic compound is present in an amount of from 0% to 50%, typically from 0.1% to 40%, more typically 0.5% to 30%, still more typically 1% to 10%, by weight relative to the total weight of the fragrance composition.

In an embodiment, the at least one basic compound is a compound represented by formula (II), or a salt thereof,

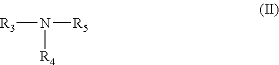

wherein
R₃ is H, aryl, C₁-C₂₀ alkyl, typically C₁-C₁₀ alkyl, more typically, C₁-C₆ alkyl, or CHRₐCOOR_{b}, wherein Rₐ and R_{b} are each, independently, H, C₁-C₂₀ alkyl, typically C₁-C₁₀ alkyl, more typically, C₁-C₆ alkyl;
R₄ and R₅ are each, independently, H, aryl, C₁-C₂₀ alkyl, typically C₁-C₁₀ alkyl, more typically, C₁-C₆ alkyl, or CHRₐCOOR_{b}, wherein Rₐ and R_{b} are each, independently, H, C₁-C₂₀ alkyl, typically C₁-C₁₀ alkyl, more typically, C₁-C₆ alkyl; or
R₄ and R₅ together form a 5- or 6-membered carbocyclic ring, optionally interrupted by one or more heteroatoms;
wherein R₃, R₄, and R₅ may optionally comprise one or more substituents selected from the group consisting of halogens, aryl, alkoxy, CN, NO₂, OH, SH, SCH₃, NH₂, COOH, and CONH₂.

Exemplary compounds represented by formula (II) include, but are not limited to, primary, secondary, and tertiary amines, carbocyclic amines, amino acids, and the like.

While the pKa of the basic compound is not particularly limited, the pKa may be considered for optimizing catalytic efficiency. In an embodiment, the at least one basic compound has a pKa>9.

In an embodiment, the at least one basic compound is selected from the group consisting of pyrrolidine, ethanolamine, diethylamine, triethylamine, tributylamine, piperidine, alanine, proline, dimethylalkylamines, diethanolalkylamines, and mixtures thereof. One or more of the basic compounds may be used in any combination. As used herein, dimethylalkylamines refer to compounds represented by formula (II) in which R₄ and R₅ are each methyl and R₃ is a C₈-C₁₆ alkyl group, such as C₈, C₁₀, C₁₂, C₁₄, or C₁₆ alkyl group. Similarly, diethanolalkylamines refer to compounds represented by formula (II) in which R₄ and R₅ are each OH-substituted ethyl groups and R₃ is a C₈-C₁₆ alkyl group, such as C₈, C₁₀, C₁₂, C₁₄, or C₁₆ alkyl group.

In some embodiments, the at least one basic compound is selected from the group consisting of ethanolamine, diethylamine, tributylamine, piperidine, alanine, proline, dimethylalkylamines, diethanolalkylamines, and mixtures thereof.

The weight ratio of the at least one 1,3-dicarbonyl compound to the at least one basic compound is not particularly limited. In an embodiment, the weight ratio of the at least one 1,3-dicarbonyl compound to the at least one basic compound is from 50:50 to 99:1, typically 80:10 to 95:5, more typically 85:15 to 95:5.

In some embodiments, the weight ratio of the at least one 1,3-dicarbonyl compound to the at least one basic compound is from 60:40 to 99:1, typically 85:15 to 98:2.

The composition according to the present disclosure includes at least one ingredient selected from the group consisting of a perfumery carrier and a perfumery base, wherein the at least one ingredient is different from the at least one 1,3-dicarbonyl compound.

As used herein, “perfumery carrier” refers to a material which is practically neutral from a perfumery point of view, i.e., that it does not significantly alter the organoleptic properties of perfuming ingredients. Said carrier may be a liquid or a solid. Exemplary liquid carriers include, but are not limited to, an emulsifying system, i.e., a solvent and a surfactant system, or a solvent typically used in perfumery. A detailed description of the nature and type of solvents commonly used in perfumery cannot be exhaustive. However, suitable solvents include, but are not limited to, glycols, such as propylene or butylene glycol; glycerol; dipropyleneglycol and its monoether, 1,2,3-propanetriyl triacetate, dimethyl glutarate, dimethyl adipate, 1,3-diacetyloxypropan-2-yl acetate, diethyl phthalate, isopropyl myristate, rosin resins (such as Abalyn® available from Eastman), benzyl benzoate, benzyl alcohol, 2-(2-ethoxyethoxy)-1-ethanol, tri-ethyl citrate, and mixtures thereof; and naturally derived solvents, such as glycerol and vegetable oils, including palm oil, sunflower oil, linseed oil, and mixtures thereof.

Solid carriers are materials to which the fragrance composition or some element of the fragrance composition can be chemically or physically bound. In general, such solid carriers are employed either to stabilize the composition, or to control the rate of evaporation of the compositions or of some ingredients. Solid carriers are of current use in the art and a person skilled in the art knows how to reach the desired effect. Suitable solid carriers include, but are not limited to, absorbing gums or polymers or inorganic materials, such as porous polymers, cyclodextrines, dextrines, maltodextrines wood-based materials, organic or inorganic gels, clays, gypsum talc or zeolites.

Other suitable solid carriers include encapsulating materials. Examples of such materials may comprise wall-forming and plasticizing materials, such as glucose syrups, natural or modified starches, hydrocolloids, cellulose derivatives, polyvinyl acetates, polyvinylalcohols, proteins or pectins, plant gums such as acacia gum (Gum Arabic), urea, sodium chloride, sodium sulphate, zeolite, sodium carbonate, sodium bicarbonate, clay, talc, calcium carbonate, magnesium sulfate, gypsum, calcium sulfate, magnesium oxide, zinc oxide, titanium dioxide, calcium chloride, potassium chloride, magnesium chloride, zinc chloride, carbohydrates, saccharides such as sucrose, mono-, di-, and polysaccharides and derivatives such as chitosan, starch, cellulose, carboxymethyl methylcellulose, methylcellulose, hydroxyethyl cellulose, ethyl cellulose, propyl cellulose, polyols/sugar alcohols such as sorbitol, maltitol, xylitol, erythritol, and isomalt, polyethylene glycol (PEG), polyvinyl pyrrolidin (PVP), polyvinyl alcohol, acrylamides, acrylates, polyacrylic acid and related, maleic anhydride copolymers, amine-functional polymers, vinyl ethers, styrenes, polystyrenesulfonates, vinyl acids, ethylene glycol-propylene glycol block copolymers, vegetable gums, gum acacia, pectins, xanthanes, alginates, carragenans, citric acid or any water soluble solid acid, fatty alcohols or fatty acids and mixtures thereof.

Other suitable encapsulating materials are described in reference texts known to those of skill in the art, such as H. Scherz, Hydrokolloide: Stabilisatoren, Dickungs-und Geliermittel in Lebensmitteln, Band 2 der Schriftenreihe Lebensmittelchemie, Lebensmittelqualität, Behr's Verlag Gmbh & Co., Hamburg, 1996. The encapsulation is a well-known process to a person skilled in the art, and may be performed, for instance, by using techniques such as spray-drying, agglomeration or yet extrusion; or consists of a coating encapsulation, including coacervation and complex coacervation techniques.

Other exemplary solid carriers include core-shell capsules with resins of aminoplast, polyamide, polyester, polyurea or polyurethane type, and mixtures thereof, made using techniques well-known to those of ordinary skill in the art, such as phase separation induced by polymerization, interfacial polymerization, coacervation, or a combination thereof, optionally in the presence of a polymeric stabilizer or of a cationic copolymer.

Resins may be produced by the polycondensation of an aldehyde (e.g., formaldehyde, 2,2-dimethoxyethanal, glyoxal, glyoxylic acid or glycolaldehyde, and mixtures thereof) with an amine such as urea, benzoguanamine, glycoluryl, melamine, methylol melamine, methylated methylol melamine, guanazole and the like, as well as mixtures thereof. Alternatively, one may use preformed resins like alkylolated polyamines such as those commercially available under the trademark Urac® (origin: Cytec Technology Corp.), Cymel® (origin: Cytec Technology Corp.), Urecoll® or Luracoll® (origin: BASF).

Other suitable resins are the those produced by the polycondensation of a polyol, like glycerol, and a polyisocyanate, for example, a trimer of hexamethylene diisocyanate, a trimer of isophorone diisocyanate or xylylene diisocyanate or a Biuret of hexamethylene diisocyanate or a trimer of xylylene diisocyanate with trimethylolpropane (marketed as Takenate® by Mitsui Chemicals), among which a trimer of xylylene diisocyanate with trimethylolpropane and a Biuret of hexamethylene diisocyanate are preferred.

The encapsulation of perfumes by polycondensation of amino resins, namely melamine-based resins with aldehydes is well-known in the art. Pertinent publications include, but are not limited to, K. Dietrich et al. Acta Polymerica, 1989, vol. 40, pages 243, 325 and 683, as well as 1990, vol. 41, page 91 and U.S. Pat. No. 4,396,670 issued Aug. 2, 1983. The general knowledge in encapsulation technology is very significant and cannot be exhaustive. More recent publications of pertinence, which disclose suitable uses of such microcapsules, are represented, for example, by the article of K. Bruyninckx and M. Dusselier, ACS Sustainable Chemistry & Engineering, 2019, vol. 7, pages 8041-8054. These publications are incorporated herein by reference.

The perfumery base is a composition comprising at least one perfuming co-ingredient. According to the present disclosure, the perfuming co-ingredient is different from the at least one 1,3-dicarbonyl compound and is a compound that imparts a hedonic effect, i.e., used for the primary purpose of conferring or modulating an odor. In other words, such a co-ingredient, to be considered a perfuming co-ingredient, must be recognized by a person skilled in the art as being able to impart or modify in a positive or pleasant way the odor of a composition, and not just as having an odor. In some embodiments, the perfuming co-ingredient may impart an additional benefit beyond that of modifying or imparting an odor, such as long-lasting, blooming, malodour counteraction, antimicrobial effect, antiviral effect, microbial stability, or pest control.

The nature and type of the perfuming co-ingredients present in the base do not warrant a more detailed description here, which in any case would not be exhaustive. Those of ordinary skill in the art are able to select them on the basis of general knowledge and according to the intended use or application and the desired organoleptic effect. In general terms, perfuming co-ingredients belong to chemical classes as varied as alcohols, lactones, esters, ethers, acetates, nitriles, terpenoids, nitrogenous or sulphurous heterocyclic compounds and essential oils, and said perfuming co-ingredients can be of natural or synthetic origin. Suitable perfuming co-ingredients include compounds known to release various types of perfuming compounds in a controlled manner, typically known as properfume or profragrance. The perfumery base according to the present disclosure is not limited to the above mentioned perfuming co-ingredients, and many other suitable co-ingredients are listed in reference texts known to those of ordinary skill in the art, such as, for example, the book by S. Arctander, Perfume and Flavor Chemicals, 1969, Montclair, New Jersey, USA, or its more recent versions, which is incorporated herein reference.

For the compositions which comprise both a perfumery carrier and a perfumery base, other suitable perfumery carriers than those previously specified, may be ethanol, water/ethanol mixtures, limonene or other terpenes, isoparaffins such as those known under the trademark Isopar® (available from Exxon Chemical) or glycol ethers and glycol ether esters, such as those known under the trademark Dowanol® (available from Dow Chemical Company), or hydrogenated castors oils such as those known under the trademark Cremophor® RH 40 (available from BASF).

The fragrance composition may optionally comprise at least one perfumery adjuvant.

The at least one perfumery adjuvant is an ingredient capable of imparting an additional added benefit such as a color, a particular light resistance, chemical stability, etc. A detailed description of the nature and type of adjuvant commonly used in perfuming composition cannot be exhaustive, but it has to be mentioned that said ingredients are well known to a person skilled in the art.

Exemplary perfumery adjuvants include, but are not limited to, viscosity agents (e.g., surfactants, thickeners, gelling and/or rheology modifiers), stabilizing agents (e.g., preservatives, antioxidant, heat/light and or buffers or chelating agents, such as BHT), coloring agents (e.g., dyes and/or pigments), preservatives (e.g. antibacterial or antimicrobial or antifungal or anti irritant agents), abrasives, skin cooling agents, fixatives, insect repellants, ointments, vitamins and mixtures thereof.

Fixatives, also known as “modulators”, are agents having the capacity to affect the manner in which the odor, and in particular the evaporation rate and intensity, of the compositions incorporating said modulator can be perceived by an observer or user thereof, over time, as compared to the same perception in the absence of the modulator. In particular, the modulator allows prolonging the time during which their fragrance is perceived. Suitable modulators include, but are not limited to, methyl glucoside polyol; ethyl glucoside polyol; propyl glucoside polyol; isocetyl alcohol; PPG-3 myristyl ether; neopentyl glycol diethylhexanoate; sucrose laurate; sucrose dilaurate, sucrose myristate, sucrose palmitate, sucrose stearate, sucrose distearate, sucrose tristearate, hyaluronic acid disaccharide sodium salt, sodium hyaluronate, propylene glycol propyl ether; dicetyl ether; polyglycerin-4 ethers; isoceteth-5; isoceteth-7, isoceteth-10; isoceteth-12; isoceteth-15; isoceteth-20; isoceteth-25; isoceteth-30; disodium lauroamphodipropionate; hexaethylene glycol monododecyl ether; and their mixtures; neopentyl glycol diisononanoate; cetearyl ethylhexanoate; panthenol ethyl ether, DL-panthenol, N-hexadecyl n-nonanoate, noctadecyl n-nonanoate, a profragrance, cyclodextrin, an encapsulation, and any combination thereof.

The fragrance compositions according to the present disclosure may be prepared according to any method known to those of ordinary skill in the art. The ordinarily-skilled artisan is perfectly able to design optimal formulations for the desired effect by admixing the above mentioned components to arrive at the desired fragrance composition by applying standard knowledge and concepts known to those of ordinary skill and by utilizing routine optimization methodologies.

In the second aspect, the present disclosure relates to a perfumed consumer product comprising the fragrance composition described herein.

The perfumed consumer product may be:
a perfume, such as a fine perfume, a splash, an eau de toilette, an eau de parfum, a cologne, a shave or after-shave lotion;
a fabric care product, such as a liquid or solid detergent, detergent tablets, a detergent bar, a detergent paste, a detergent pouch, a liquid fabric softener, fabric softener sheets, a fabric scent booster, a laundry pre-treatment, a fabric refresher, an ironing water, or a laundry bleach,
a hair care product, such as a shampoo, a hair conditioner, such as rinse-off conditioner or leave-on conditioner; a hair cream, a hair oil, a hair styling product, such as a spray, mousse, or gel; a hair coloration product, or a hair permanent wave product;
a skin care product, such as a face cream, a body-or-face cream, a face lotion, a shaving product (which may be in the form of a foam, cream, gel or oil), a body and/or hand product (which may be in the form of a lotion, cream, gel or oil), a skin firming product, a depilatory, a talcum powder, a foot care cream or lotion, baby wipes, cleansing wipes, moisturizer wipes, a tanning or sun-protection product (which may be in the form of a spray, lotion, cream or oil), a foot/hand care product, a makeup, a vanishing cream;
a skin-cleansing product, such as a soap bar, a shower gel, a liquid hand soap, a bath foam, a shower or bath mousse, oil or gel; body scrub, a hygiene product, or an intimate wash product;
a body deodorant or antiperspirant product, such as a body deodorant spray, a roll-on deodorant, a deodorant stick, a deodorant cream, an antiperspirant spray, an antiperspirant stick, a roll-on antiperspirant liquid, an antiperspirant stick, or an antiperspirant cream;
a nail product;
a home care product, such as a disinfectant, a mold remover, a furniture care product, a hard-surface wipe, a dish detergent, a hard-surface detergent, a leather care product, or a car care product.

In an embodiment, the perfumery consumer product is a hair care product selected from the group consisting of a shampoo, rinse-off conditioner, and leave-on conditioner.

In another embodiment, the perfumery consumer product is a skin care and/or a skin cleansing product selected from the group consisting of a body cream, a face cream, a body-or-face cream, a shower gel, a soap bar, and a body scrub.

The amount of the fragrance composition used in the perfumed consumer product is not particularly limited. However, in an embodiment, the amount of the fragrance composition is from 0.25% to 5%, typically 0.50 to 2%, more typically 0.75% to 1.5%, by weight relative to the total amount of the perfumed consumer product.

The amount of the at least one 1,3-dicarbonyl compound in the perfumed consumer product is not particularly limited. In an embodiment, the amount of the at least one 1,3-dicarbonyl compound is from 0.01% to 4%, typically 0.05% to 2%, more typically 0.1% to 1.5%, by weight relative to the total amount of the perfumed consumer product.

The amount of the at least one basic compound in the perfumed consumer product is not particularly limited. However, in an embodiment, the amount of the at least one basic compound is from 0.0001% to 2%, typically 0.0025% to 1%, more typically 0.0025% to 0.5%, still more typically 0.02% to 0.3%, by weight relative to the total amount of the perfumed consumer product.

In the third aspect, the present disclosure relates to use of the fragrance composition or perfumed consumer product described herein to prevent, reduce, or ameliorate malodorous aldehydes and ketones from the hair, scalp, skin, or any combination thereof, of a subject.

The use of the fragrance composition or perfumed consumer product described herein to prevent, reduce, or ameliorate malodorous aldehydes and ketones from the hair, scalp, skin, or any combination thereof, of a subject may be accomplished using methods known to those of ordinary skill in the art. In one suitable method, the method comprises applying a fragrance composition or a perfumed consumer product described herein to the hair, scalp, skin, or any combination thereof, of a subject, typically a human being. In an embodiment, the method comprises massaging the fragrance composition or the perfumed consumer product described herein to the hair or scalp of a subject.

In the fourth aspect, the present disclosure relates to use of the fragrance composition or perfumed consumer product described herein to prevent, reduce, or ameliorate malodorous aldehydes and ketones from any inanimate surface.

Malodor may arise from inanimate surfaces, such as fabrics and textiles used in clothing, bedding, upholstery, and the like, due to transfer of sweat, lipids, and/or oils from the skin of a user. By autoxidation, bacterial action, or enzymatic action, the sweat, lipids, and/or oils which become deposited onto the surface can be oxidized to intermediate hydroperoxides that further decompose to release malodorous aldehydes and other compounds. In a suitable method for reducing malodorous aldehydes and ketones from any inanimate surface, the method comprises contacting the fragrance composition or perfumed consumer product described herein to the inanimate surface.

In some aspects, degradation of lipid hydroperoxides on skin, particularly in middle aged and elderly people, releases malodorous aldehydes, primarily the compound 2-nonenal. Thus, in an embodiment of the above-mentioned uses, the malodorous aldehydes comprise 2-nonenal.

The compositions, products, uses, and methods according to the present disclosure are further illustrated by the following non-limiting examples.

### Example 1. Analytical Reduction of Aldehydes and Ketones from Scalp Malodor

In the present examples, a scalp malodor reconstitution was used to represent scalp malodor. The components of the scalp malodor reconstitution are shown in Table 1.

TABLE 1

| Ingredients | Amount (wt %) |
|---|---|
| BENZALDEHYDE | 1.30 |
| ALDEHYDE C9 | 8.00 |
| GERANYL ACETONE HLR | 9.00 |
| METHYLHEPTENONE | 13.50 |
| Q.S. to 100 with other | 68.20 |
| ingredients | |

Mixtures of the scalp malodor reconstitution, ethyl acetoacetate and various basic compounds were prepared according to Table 2 below. Amounts of each component are expressed in parts by weight.

TABLE 2

| Components | C1 | A | B | C | D | E | F | G | H | I |
|---|---|---|---|---|---|---|---|---|---|---|
| Scalp | 100 | 100 | 100 | 100 | 100 | 100 | 100 | 100 | 100 | 100 |
| Malodor | | | | | | | | | | |
| Ethyl | — | 90 | — | 90 | — | 90 | — | 90 | — | 90 |
| Acetoacetate | | | | | | | | | | |
| Ethanolamine | — | — | 10 | 10 | — | — | — | — | — | — |
| Proline | — | — | — | — | 10 | 10 | — | — | — | — |
| Diethylamine | — | — | — | — | — | — | 10 | 10 | — | — |
| Tributylamine | — | — | — | — | — | — | — | — | 10 | 10 |
| DIPG | — | 10 | 90 | 0 | 90 | 0 | 90 | 0 | 90 | 0 |

Each mixture was stirred until homogeneous and allowed to stand at room temperature for 2 hours. A color change from red-orange to yellowish was observed when the reaction was completed.

The scalp malodor reconstitution alone (comparative example C1) and the mixtures A-I were analyzed by Gas Chromatography coupled with Mass Spectrometry, GC-MS. 1 g of product was placed in a 20-ml solid-phase microextraction, SPME, vial and the degradation of the aldehydes and ketones was measured from the headspace above the mixture.

The analysis was performed at room temperature with a GC-MS (GC Agilent 5975 B) using a SPME method (1 g sample/Column DB-1 ms, length 30 m×I.D 25 mm×film 0.25 μm/Helium flow 1.2 ml/min, pressure 80.5 Kpa/Split 20/60° C. 2 min, 7° C./min until 250° C.).

The amounts of scalp malodor components remaining in mixtures A-I are shown in Tables 3a and 3b below and are expressed as wt % relative to the weight of the starting scalp malodor reconstitution.

TABLE 3a

| Scalp malodor component | C1 | A | B | C | D |
|---|---|---|---|---|---|
| Benzaldehyde | 1.3 | 1.16 | 0.009 | 0.006 | 0.87 |
| Aldehyde C9 | 8.00 | 2.72 | 0.16 | 0.00 | 1.12 |
| Methylheptenone | 13.50 | 13.50 | 10.95 | 6.05 | 11.33 |
| Geranyl Acetone | 9.00 | 8.07 | 4.12 | 3.69 | 7.26 |

TABLE 3b

| Scalp malodor component | E | F | G | H | I |
|---|---|---|---|---|---|
| Benzaldehyde | 0.28 | 0.69 | 0.030 | 0.84 | 0.71 |
| Aldehyde C9 | 0.055 | 1.37 | 0.0072 | 6.07 | 0.29 |
| Methylheptenone | 8.27 | 10.65 | 10.55 | 10.76 | 8.68 |
| Geranyl Acetone | 4.84 | 7.26 | 6.04 | 7.66 | 4.27 |

While the analytical GC-MS results confirmed that sample A containing only ethyl acetoacetate (no basic compound) and samples containing only the basic compound (B, D, F, H) could already reduce the level of aldehydes and ketones present in the scalp malodor, with the best impact on aldehyde C9, a significantly better reduction of both aldehydes and ketones is obtained with the combination of ethyl acetoacetate and a basic compound, best results being achieved with ethanolamine as the basic compound. In this reaction the basic compound enhances the efficiency of the reaction between the active and the malodorous aldehydes and ketones.

### Example 2. Olfactive Evaluation

The samples prepared according to Example 1 were olfactively evaluated out of a jar by an expert panel of 5 people. An evaluation scale between 1-7 was used (1=no malodor//7=very intense malodor). The results of the olfactive evaluation are reported in Table 4 below.

TABLE 4

| Samples | Malodor Intensity (1-7) |
|---|---|
| C1 | 7 |
| A | 6 |
| B | 5 |
| C | 2 |
| D | 5 |
| E | 2.5 |
| F | 5 |
| G | 2.5 |
| H | 5 |
| I | 3 |

It has been demonstrated that the perception of scalp malodor was reduced by using either ethyl acetoacetate alone (A) or the basic compound alone (B, D, F, H). However, the combination of ethyl acetoacetate and basic compound was far superior (C vs. A and B and C1, E vs. A and D and C1, G vs. A and F and C1, I vs. A and H and C1).

Thus, the analytical results from Table 3 are in accordance with the olfactive evaluations summarized in Table 4.

### Example 3. Efficiency of Scalp Malodor Reduction

Tests of mixtures of ethyl acetoacetate and ethanolamine at varying concentrations were conducted to determine an optimal efficiency for reducing scalp malodor. A number of mixtures of the scalp malodor reconstitution (Table 1), ethyl acetoacetate and ethanolamine were prepared in which the amounts of each component were varied. The mixtures were stirred until homogeneous and allowed to stand at room temperature for 2 hours. A color change from red-orange to yellowish was observed when the reaction was completed. The samples and component amounts are summarized in Table 5 below, with amounts expressed in parts by weight.

TABLE 5

| Components | C1 | C | J | K | L | M | N | O | P | Q | R | S |
|---|---|---|---|---|---|---|---|---|---|---|---|---|
| Scalp | 100 | 100 | 100 | 100 | 100 | 100 | 100 | 100 | 100 | 100 | 100 | 100 |
| Malodor | | | | | | | | | | | | |
| Ethyl | 0 | 90 | 90 | 90 | 90 | 80 | 50 | 50 | 30 | 30 | 30 | 10 |
| Acetoacetate | | | | | | | | | | | | |
| Ethanolamine | 0 | 10 | 5 | 1 | 0.5 | 10 | 10 | 2 | 10 | 5 | 2 | 10 |
| DIPG | 0 | 0 | 5 | 9 | 9.5 | 10 | 40 | 48 | 60 | 65 | 68 | 80 |

Samples J-S were analyzed by GC-MS according to the procedure described in Example 1. The results are summarized in Tables 6a and 6b below. The amounts of scalp malodor components remaining in mixtures J-S are expressed as wt % relative to the weight of the starting scalp malodor reconstitution.

TABLE 6a

| Scalp malodor | | | | | | |
|---|---|---|---|---|---|---|
| components | C1 | C | J | K | L | M |
| Benzaldehyde | 1.3 | 0.006 | 0.0065 | 0.53 | 0.56 | 0.0062 |
| Aldehyde C9 | 8 | 0.00 | 0.015 | 0.47 | 0.54 | 0.013 |
| Methylheptenone | 13.50 | 6.05 | 6.75 | 8.41 | 8.82 | 6.74 |
| Geranyl Acetone | 9 | 3.69 | 4.63 | 5.19 | 5.27 | 4.54 |

TABLE 6b

| Scalp malodor | | | | | | |
|---|---|---|---|---|---|---|
| components | N | O | P | Q | R | S |
| Benzaldehyde | 0.0072 | 0.052 | 0.0074 | 0.060 | 0.20 | 0.067 |
| Aldehyde C9 | 0.013 | 0.13 | 0.057 | 0.32 | 0.43 | 0.022 |
| Methylheptenone | 9.12 | 9.27 | 9.22 | 9.40 | 9.74 | 12.96 |
| Geranyl Acetone | 6.41 | 7.01 | 7.30 | 8.43 | 8.50 | 8.50 |

It was observed that a decrease of the active compound, ethyl acetoacetate, or the basic compound, ethanolamine, dosages resulted in a lowered reduction of the malodorous aldehydes and ketones from the scalp.

Comparing the results obtained with 0.9% versus 0.1% of ethyl acetoacetate in the final application, it was observed that 0.1% of ethyl acetoacetate reduced less the concentrations of malodorous aldehydes and ketones. A reduction of the aldehydes and ketones from scalp malodor was still noticed but to a lesser extent. The concentration of the remaining aldehydes has almost not changed but the concentration of the remaining ketones has significantly increased (Sample C vs. S).

Comparing the results obtained with 0.1% versus 0.005% of ethanolamine in the final application, it was observed that 0.005% of ethanolamine had less impact on the reduction of the aldehydes and ketones concentrations. A reduction of the aldehydes and ketones from scalp malodor was still apparent but to a lesser extent, especially on the reduction of benzaldehyde. The concentration of the remaining aldehyde C9 has almost not changed, the concentration of the remaining ketones has slightly increased and the concentration of the remaining benzaldehyde has significantly increased (Sample C vs. L).

### Example 4. Leave-on Conditioner

A leave-on hair conditioner composition was prepared using the components and amounts summarized in Table 7 below.

TABLE 7

| Phase | Ingredients/INCI name | Amount (wt %) |
|---|---|---|
| A | Water deionized | 88.575 |
| | Tetrasodium EDTA ¹⁾ | 0.05 |
| | Guar | 0.30 |
| | Hydroxypropyltrimonium | |
| | Chloride ²⁾ | |
| B | Paraffin Liquid, pure ³⁾ | 2.00 |
| C | Cetrimonium Chloride ⁴⁾ | 1.00 |
| D | Cetearyl Alcohol (and) | 5.00 |
| | Dipalmitoylethyl | |
| | Hydroxyethylmonium | |
| | Methosulfate (and) | |
| | Ceteareth-20 ⁵⁾ | |
| E | Glycerin ⁶⁾ | 2.00 |
| F | Amodimethicone (and) | 1.00 |
| | trideceth-12 (and) | |
| | Cetrimonium Chloride ⁷⁾ | |
| G | Methylchloroisothiazolinone | 0.075 |
| | (and) Methylisothiazolinone ⁸⁾ | |
| | TOTAL: | 100 |

¹⁾ EDTA B POWDER, origin: BASF
²⁾ JAGUAR C14 S, origin: Lubrizol
³⁾ PARAFFINUM PERLIQUIDUM, origin: Acros Organics
⁴⁾ GENAMIN CTAC, origin: Clariant
⁵⁾ DEHYQUART C 4046, origin: BASF
⁶⁾ GLYCERIN 85%, origin: Brenntag
⁷⁾ XIAMETER MEM-949 Cationic Emulsion, origin: Xiameter
⁸⁾ KATHON CG, origin: Sigma-Aldrich

Tetrasodium EDTA was dissolved in water prior to the addition of guar while stirring until it was fully dispersed. Paraffine was added and the mixture was stirred until it became slightly creamy. Genamin CTAC was added and the mixture was stirred until well homogenized. Then the mixture was heated to 75° C. While stirring, Phase C was added and then the mixture was cooled down to 40° C. Phase D was added to vessel and stirred until obtention of a homogenized mixture. The mixture started to be emulsified. While emulsifying the mixture, Phase E and F were added. The emulsifying process lasted until obtention of a cream. The mixture was cooled down to 35° C. and the preservative was added. The emulsification was kept until reaching the proper viscosity of the product. A mixture of ethyl acetoacetate/ethanolamine (90/10) was prepared and applied at 0.50% in the leave-on conditioner.

0.1 g of this leave-on conditioner was applied on a hair swatch (0.5 g/8 cm×1 cm) and the hair swatch was gently massaged between 2 fingers until the product was well absorbed by the hair. The same hair swatch was then contaminated with 0.050 g of the scalp malodor reconstitution (Table 1) diluted at 1% in isopropyl myristate. The hair swatch was gently massaged between 2 fingers until the product was well absorbed by the hair and then cut in 3 pieces and placed on a 20 ml SPME vial. The vial containing the hair swatch was closed and left at room temperature for 24 hours to allow the reaction to occur and then analyzed by GC-MS according to the procedure described in Example 1. This procedure was carried out with 3 hair swatches and the average results are reported in Table 8. The amounts of scalp malodor components remaining on the hair swatches are expressed as wt % relative to the weight of the starting scalp malodor reconstitution.

TABLE 8

| | Scalp malodor | |
|---|---|---|
| Components | reconstitution only | Leave-On Conditioner |
| Benzaldehyde | 1.30 | 0.90 |
| Aldehyde C9 | 8.00 | 3.69 |
| Methylheptenone | 13.50 | 7.54 |
| Geranyl Acetone | 9.00 | 5.75 |

The measurements summarized in Table 8 show that a leave-on conditioner, containing 0.45% of ethyl acetoacetate and 0.05% of ethanolamine, when deposited on hair, had a very good impact on the reduction of the aldehydes and ketones present in the scalp malodor reconstitution. A reduction of almost half of the aldehydes and the ketones concentration was measured. Furthermore, amongst the aldehydes and ketones present in the scalp malodor reconstitution, aldehyde C9 and methylheptenone were reduced most efficiently.

### Example 5. Rinse-Off Conditioner

A rinse-off hair conditioner composition was prepared using the components and amounts summarized in Table 9 below.

TABLE 9

| | | Concentration |
|---|---|---|
| Phase | Ingredients | (wt %) |
| A | Water deionized | 81.8 |
| | Behentrimonium Chloride ¹⁾ | 2.5 |
| | Hydroxyethylcellulose ²⁾ | 1.5 |
| B | Cetearyl Alcohol ³⁾ | 4 |
| | Glyceryl Stearate (and) PEG-100 Stearate ⁴⁾ | 2 |
| | Behentrimonium Methosulfate (and) Cetyl alcohol | 4 |
| | (and) Butylene Glycol ⁵⁾ | |
| | Ethoxy (20) Stearyl Alcohol ⁶⁾ | 1 |
| C | Amodimethicone (and) Trideceth-12 (and) | 3 |
| | Cetrimonium Chloride ⁷⁾ | |
| | Chlorhexidine Digluconate ⁸⁾ 20% aqueous | 0.2 |
| | solution | |
| D | Citric acid 10% aqueous sol. till pH 3.5-4 | q.s. |
| | TOTAL: | 100 |

¹⁾ Genamin KDMP, Clariant
²⁾ Tylose H10 Y G4, Shin Etsu
³⁾ Lanette O, BASF
⁴⁾ Arlacel 165, Croda
⁵⁾ Incroquat Behenyl TMS-50-PA- (MH), Croda
⁶⁾ Brij S20, Croda
⁷⁾ Xiameter MEM-949, Dow Corning
⁸⁾ Alfa Aesar

Ingredients of Phase A were mixed until a uniform mixture was obtained. Tylose was allowed to completely dissolve. Then the mixture was heated to 70-75° C. Ingredients of Phase B were combined and melted at 70-75° C. Then ingredients of Phase B were added to Phase A with good agitation and the mixing was continued until cooled down to 60° C. Then, ingredients of Phase C were added while agitating and continuously mixing until the mixture cooled down to 40° C. The pH was adjusted with citric acid solution until pH 3.5-4.0. A mixture of ethyl acetoacetate/ethanolamine (90/10) was prepared and applied at 0.50% in the rinse-off conditioner.

0.1 g of this rinse-off conditioner was applied on a hair swatch (0.5 g/8 cm×1 cm) and the hair swatch was gently massaged between 2 fingers until the product was well absorbed by the hair. Then it was dipped 3 times (3 times in, 3 times out) into a beaker filled with 200 ml of warm water. Then dipped again into the same beaker and slowly moved back and forth 3 times in each direction. The same rinse protocol was repeated with a second beaker filled with 200 mL of warm water. The excess of water from the hair swatch was squeezed between 2 fingers. The same hair swatch was contaminated with 0.050 g of the scalp malodor reconstitution (Table 1) diluted at 1% in isopropyl myristate. The hair swatch was gently massaged between 2 fingers until the product was well absorbed by the hair and then cut in 3 pieces and placed on a 20 ml SPME vial. The vial containing the hair swatch was closed and left at room temperature for 24 hours to allow the reaction to occur and then analyzed by GC-MS according to the procedure described in Example 1. This procedure was carried out with 3 hair swatches and the average results are reported in Table 10. The amounts of scalp malodor components remaining on the hair swatches are expressed as wt % relative to the weight of the starting scalp malodor reconstitution.

TABLE 10

| | Scalp malodor | |
|---|---|---|
| Components | reconstitution only | Rinse-Off Conditioner |
| Benzaldehyde | 1.3 | 0.93 |
| Aldehyde C9 | 8 | 4.67 |
| Methylheptenone | 13.5 | 8.21 |
| Geranyl Acetone | 9 | 7.08 |

The measurements summarized in Table 10 show that a rinse-off conditioner, containing 0.45% of ethyl acetoacetate and 0.05% of ethanolamine, when deposited on hair, had a positive impact on the reduction of the aldehydes and ketones present in the scalp malodor reconstitution. A reduction of almost one-third of the aldehydes and the ketones concentration was measured. Furthermore, amongst the aldehydes and ketones present in scalp malodor reconstitution, aldehyde C9 and methylheptenone were reduced most efficiently.

### Example 6. Shampoo Composition

A pearly shampoo composition was prepared using the components and amounts summarized in Table 11 below.

TABLE 11

| | | Concentration |
|---|---|---|
| Phase | Ingredients | (wt %) |
| A | Water deionized | 45.97 |
| | Tetrasodium EDTA ¹⁾ | 0.05 |
| | Guar Hydroxypropyltrimonium Chloride ²⁾ | 0.05 |
| | Polyquaternium-10 ³⁾ | 0.075 |
| B | NaOH 10% aqueous sol. | 0.3 |
| C | Ammonium Lauryl Sulfate ⁴⁾ | 34 |
| | Ammonium Laureth Sulfate ⁵⁾ | 9.25 |
| | Cocamidopropyl Betaine ⁶⁾ | 2 |
| | Dimethicone (and) C12-13 Pareth-4 (and) C12-13 | 2.5 |
| | Pareth-23 (and) Salicylic Acid ⁷⁾ | |
| D | Cetyl Alcohol ⁸⁾ | 1.2 |
| | Cocamide MEA ⁹⁾ | 1.5 |
| | Glycol Distearate ¹⁰⁾ | 2 |
| E | Methylchloroisothiazolinone (and) | 0.1 |
| | Methylisothiazolinone ¹¹⁾ | |
| | D-Panthenol 75% ¹²⁾ | 0.1 |
| | Water deionized | 0.3 |
| F | Sodium Chloride 25% aqueous sol. | 0.6 |
| | TOTAL: | 100 |

¹⁾ EDETA B Powder, BASF
²⁾ Jaguar C14 S, Rhodia
³⁾ Ucare Polymer JR-400, Noveon
⁴⁾ Sulfetal LA B-E, Zschimmer & Schwarz
⁵⁾ Zetesol LA, Zschimmer & Schwarz
⁶⁾ Tego Betain F 50, Evonik
⁷⁾ Xiameter MEM-1691, Dow Corning
⁸⁾ Lanette 16, BASF
⁹⁾ Comperlan 100, Cognis
¹⁰⁾ Cutina AGS, Cognis
¹¹⁾ Kathon CG, Rohm & Haas
¹²⁾ D-Panthenol, Roche

A premix having Guar Hydroxypropyltrimonium Chloride and Polyquaternium-10 was added to water and Tetrasodium EDTA while mixing. When the mixture was homogeneous, NaOH was added. Then, Phase C ingredients were added and the mixture was heated to 75° C. Phase D ingredients were added and mixed until homogeneous. The heating was stopped, and the temperature of the mixture was decreased to RT. At 45° C., ingredients of Phase E were added while mixing. The final viscosity was adjusted with 25% NaCl solution and pH of 5.5-6 was adjusted with 10% NaOH solution. A mixture of ethyl acetoacetate/ethanolamine (90/10) was prepared and applied at 0.50% in the pearly shampoo.

0.1 g of this shampoo was applied on a hair swatch (0.5 g/8 cm×1 cm) and the hair swatch was gently washed between 2 fingers until the product was well foaming. Then it was dipped 3 times (3 times in, 3 times out) into a beaker filled with 200 ml of warm water. Then dipped again into the same beaker and slowly moved back and forth 3 times in each direction. The same rinse protocol was repeated with a second beaker filled with 200 mL of warm water. The excess of water from the hair swatch was squeezed between 2 fingers. The same hair swatch was contaminated with 0.050 g of a scalp malodor reconstitution (Table 1) diluted at 1% in isopropyl myristate. The hair swatch was gently massaged between 2 fingers until the product was well absorbed by the hair and then cut in 3 pieces and placed on a 20 ml SPME vial. The vial containing the hair swatch was closed and left at room temperature for 24 hours to allow the reaction to occur and then analyzed by GC-MS according to the procedure described in Example 1. This procedure was carried out with 3 hair swatches and the average results are reported in Table 12 below. The amounts of scalp malodor components remaining on the hair swatches are expressed as wt % relative to the weight of the starting scalp malodor reconstitution.

TABLE 12

| | Scalp malodor | |
|---|---|---|
| Components | reconstitution only | Shampoo |
| Benzaldehyde | 1.3 | 1.16 |
| Aldehyde C9 | 8 | 7.15 |
| Methylheptenone | 13.5 | 11.01 |
| Geranyl Acetone | 9 | 8.86 |

The measurements summarized in Table 12 show that a shampoo, containing 0.45% of ethyl acetoacetate and 0.05% of ethanolamine, when deposited on hair, had a positive impact, albeit slight, on the reduction of the aldehydes and ketones present in the scalp malodor reconstitution. A reduction of less than 20% of the aldehydes and the ketones concentration was measured. Furthermore, amongst the aldehydes and ketones present in scalp malodor reconstitution, aldehyde C9 and methylheptenone were reduced most efficiently.

### Example 7. Olfactive Evaluation

Mixtures of ethyl acetoacetate/ethanolamine (90/10) were incorporated into hair care applications at various concentrations (0.1%, 0.3%, and 0.5% by weight), forming samples T-AB.

Hair swatches (10 g/20 cm×7 cm) were pre-treated with 1 g of each of the samples T-AB and then contaminated with 0.50 g the scalp malodor reconstitution (Table 1) diluted at 1% in isopropyl myristate using the same protocol described in Examples 4, 5, and 6. The hair swatches were then olfactively evaluated by an expert panel of 5 people. An evaluation scale between 1-7 was used (1=no malodor//7=very intense malodor). The results of the olfactive evaluation are reported in Table 13 below.

TABLE 13

| | | Concentration in | | |
|---|---|---|---|---|
| | | application - | Malodor | |
| | | sample/(ethyl | Intensity (1-7) | |
| | Applica- | acetoacetate:ethanol- | t = 30 | t = 6 |
| Samples | tion | amine) | min | H |
| Scalp malodor | — | — | 7 | 6 |
| reconstitution | | | | |
| only | | | | |
| (comparative) | | | | |
| T | Leave-On | 0.1%/(0.09%:0.01%) | 4.5 | 4 |
| | Conditioner | | | |
| U | Leave-On | 0.3%/(0.27%:0.03%) | 4 | 3 |
| | Conditioner | | | |
| V | Leave-On | 0.5%/(0.45%:0.05%) | 3 | 2 |
| | Conditioner | | | |
| W | Rinse-Off | 0.1%/(0.09%:0.01%) | 6 | 5 |
| | Conditioner | | | |
| X | Rinse-Off | 0.3%/(0.27%:0.03%) | 5 | 4 |
| | Conditioner | | | |
| Y | Rinse-Off | 0.5%/(0.45%:0.05%) | 4 | 4 |
| | Conditioner | | | |
| Z | Shampoo | 0.1%/(0.09%:0.01%) | 6.5 | 6 |
| AA | Shampoo | 0.3%/(0.27%:0.03%) | 5.5 | 5 |
| AB | Shampoo | 0.5%/(0.45%:0.05%) | 5 | 4 |

As shown in Table 13, the perception of scalp malodor was reduced by using different hair care applications containing a mixture of ethyl acetoacetate and ethanolamine. The best performance against scalp malodor was obtained when the hair were pre-treated with a leave-on conditioner containing the mixture of ethyl acetoacetate and ethanolamine, followed by the rinse-off conditioner and then the shampoo.

A concentration of 0.45% of ethyl acetoacetate in combination with ethanolamine at 0.05% in the leave-on conditioner application (Sample V) showed optimal performance against the malodorous aldehydes and ketones from scalp. The malodor reduction was well noticed olfactively with a delta of 5 between that of the reference scalp malodor intensity vs. that of Sample V.

### Example 8. Fragrance Compositions

Two fragrances were prepared. One fragrance (Fragrance A) did not contain any 1,3-dicarbonyl compound or basic compound, and one fragrance (Fragrance B) contained 51% of ethyl acetoacetate and 2% of ethanolamine. In order to avoid side reactions between ethyl acetoacetate and/or ethanolamine with other ingredients in the fragrances, the fragrances were designed free of aldehydes (0.00%) and with a very low level of ketones (1.6%). The fragrance compositions are shown in Table 14 below.

TABLE 14

| Fragrance A | | Fragrance B | |
|---|---|---|---|
| Perfumery Ingredients | Wt % | Perfumery Ingredients | Wt % |
| Methylbutyrate 2 Ethyl | 6.25 | Methylbutyrate 2 Ethyl | 6.25 |
| Ethyl Acetoacetate | 0.00 | Ethyl Acetoacetate | 51.00 |
| Ethanolamine | 0.00 | Ethanolamine | 2.00 |
| Acetate cis-3-Hexenol | 1.48 | Acetate cis-3-Hexenol | 1.48 |
| Butyrate isoamyle | 0.82 | Butyrate isoamyle | 0.82 |
| Linalol BJ | 7.81 | Linalol BJ | 7.81 |
| Allyl Amyl Glycolate | 4.27 | Allyl Amyl Glycolate | 4.27 |
| Verdox | 8.35 | Verdox | 8.35 |
| Methyl Anthranilate | 0.14 | Methyl Anthranilate | 0.14 |
| Delta Damascone | 0.15 | Delta Damascone | 0.15 |
| William's Ester | 2.94 | William's Ester | 2.94 |
| Verdyl Acetate | 0.82 | Verdyl Acetate | 0.82 |
| Neobutenone | 0.51 | Neobutenone | 0.51 |
| Verdyl Propionate | 1.76 | Verdyl Propionate | 1.76 |
| Gamma Undecalactone | 11.43 | Gamma Undecalactone | 11.43 |
| Raspberry Ketone | 0.28 | Raspberry Ketone | 0.28 |
| DIPG | 53.00 | | |
| SUM | 100.00 | SUM | 100.00 |

1:1 mixtures of the Fragrances A and 1:0.47 mixtures of the Fragrances B with the scalp malodor reconstitution (Table 1) were prepared resulting in Sample AC and Sample AD.

According to Fragrance A and Fragrance B compositions, Sample AC had neither 1,3-dicarbonyl compound nor basic compound, while Sample AD contained ethyl acetoacetate and ethanolamine. These samples were stirred until homogeneous and allowed to stand at room temperature for 2 hours. A color change from red-orange to yellowish was observed when the reaction was completed. 1 g of each sample was weighed in a 20-ml SPME vial and the degradation of the aldehydes and ketones present in the scalp malodor was measured from the headspace by GC-MS using the analytical method described previously. The analytical results are shown on Table 15. The amounts of scalp malodor components remaining in samples AC and AD are expressed as wt % relative to the weight of the starting scalp malodor reconstitution.

TABLE 15

| | Scalp malodor | | |
|---|---|---|---|
| Component | reconstitution only | Sample AC | Sample AD |
| Benzaldehyde | 1.3 | 0.82 | 0.56 |
| Aldehyde C9 | 8 | 5.88 | 0.07 |
| Methylheptenone | 13.50 | 10.01 | 8.17 |
| Geranyl Acetone | 9 | 7.89 | 4.19 |

The measurements summarized in Table 15 show that a fragrance containing 51% of ethyl acetoacetate and 2% of ethanolamine significantly reduced the aldehydes and ketones in the scalp malodor.

### Example 9. Olfactive Evaluation on Hair Swatches

Fragrance A and Fragrance B were applied in different hair care applications at the same dosage, forming Samples AC-AD (Table 16). Hair swatches (10 g/20 cm×7 cm) were pre-treated with 1 g of the samples AC-AD and then contaminated with 0.50 g a scalp malodor reconstitution (Table 1) diluted at 1% in isopropyl myristate using the same protocol described in Example 7. The hair swatches (10 g/20 cm×7 cm) have been olfactively evaluated by an expert panel of 5 people. An evaluation scale between 1-7 was used (1=no malodor//7=very intense malodor). The results of the olfactive evaluation are reported in Table 16.

TABLE 16

| | | | Malodor Intensity | | Malodor Intensity | |
|---|---|---|---|---|---|---|
| | | | (1-7) | | (1-7) | |
| | | Dosages | Fragrance A | | Fragrance B | |
| | | (%) in | (no active) | | (with active) | |
| Samples | Applications | application | t = 30 min | t = 6 H | t = 30 min | t = 6 H |
| Scalp | — | — | 7 | 7 | 7 | 7 |
| Malodor | | | | | | |
| only | | | | | | |
| AC | Leave-On | 0.5% | 2.5 | 2.5 | 1 | 1 |
| | Conditioner | | | | | |
| AD | Rinse-Off | 0.5% | 4 | 3.5 | 3 | 2.5 |
| | Conditioner | | | | | |
| AE | Shampoo | 0.5% | 6.5 | 6 | 5 | 4.5 |

It has been verified that the perception of scalp malodor was already reduced with the hair care applications containing fragrance A (no active) but the performance against scalp malodor was much better with the hair care applications containing fragrance B (with actives).

The best performance against scalp malodor was obtained when the hair was pre-treated with a leave-on conditioner containing fragrance B, followed by the rinse-off conditioner and then the shampoo.

Fragrance B applied at 0.5% in a leave-on conditioner, showed the best performance against the malodorous aldehydes and ketones from scalp (Sample AC).

The malodor reduction was well noticed olfactively with a delta of 6 (after 6 hours) between the scalp malodor intensity reference vs. Sample AC.

### Example 10. Analytical Reduction of Trans-2-Nonenal from “Old People Malodor”

A mixture (“Product A”) of trans-2-nonenal, ethyl acetoacetate and ethanolamine was prepared (100 parts trans-2-nonenal, 90 parts ethyl acetoacetate, and 10 parts ethanolamine, by weight). The mixture was stirred until homogeneous and allowed to stand at room temperature for 2 hours. A color change from red-orange to yellowish was observed when the reaction was completed.

The “old people malodor” reconstitution (trans-2-nonenal) and the mixture with ethyl acetoacetate and ethanolamine were analyzed by GC-MS. 1 g of product was placed in a 20-ml SPME vial and the degradation of the trans-2-nonenal was measured from the headspace above the mixture.

The analysis was performed at room temperature with a GC-MS (GC Agilent 5975 B) using a SPME method (1 g sample/Column DB-1 ms, length 30 m×I.D 25 mm×film 0.25 μm/Helium flow 1.2 ml/min, pressure 80.5 Kpa/Split 20/60° C. 2 min, 7° C./min until 250° C.).

The amount of remaining malodor, as measured by GC-MS, was 18.29% by weight relative to the weight of the starting “old people malodor” reconstitution. This result shows that the mixture of ethyl acetoacetate and ethanolamine (90:10) significantly reduced the trans-2-nonenal.

The samples were olfactively evaluated out of the jar by an expert panel of 5 people. An evaluation scale between 1-7 was used (1=no malodor//7=very intense malodor). The results of the olfactive analysis are reported on Table 17 below.

TABLE 17

| Samples | Malodor Intensity (1-7) |
|---|---|
| Old people malodor (reference) | 7 |
| A | 1.5 |

The malodor reduction was also well noticed olfactively (Table 17) with a delta of 5.5 between the old people malodor intensity reference vs. Product A.

### Example 11. Body Lotion Composition

A body lotion composition was prepared using the components and amounts summarized in Table 18 below.

TABLE 18

| | | Amount |
|---|---|---|
| Phase | Ingredients | (wt %) |
| A | Steareth-2 (AND) PEG-8 Distearate ₁₎ | 5.0 |
| | Cetyl alcohol | 0.5 |
| | Ceteth-20 (AND) Glyceryl stearate (AND) PEG-6 | 4.0 |
| | stearate (AND) STEARETH-20 ²⁾ | |
| | Squalan ₃₎ | 1.0 |
| | Paraffin oil ₄₎ | 2.0 |
| | Petrolatum ⁵⁾ | 5.5 |
| B | Water deionised | 75.9 |
| | Propylene glycol | 5.0 |
| C | Phenoxyethanol (AND) PIROCTONE OLAMINE ⁶⁾ | 0.6 |
| D | Sodium carbomer ⁷⁾ | 0.2 |
| E | PERFUME | 0.3 |

₁₎ Arlacel 985
²⁾ Tefose 2561
₃₎ Biolip P 90
₄₎ Mineral oil 30-40 CPS
⁵⁾ Petroleum jelly
⁶⁾ Nipaguard PO 5
⁷⁾ PNC 400

Phases A and B were heated separately to 70-75° C. Phase A was added to phase B, then vacuum was applied. The mixture was stirred and was cooled to 55° C. under stirring over 15 minutes at room temperature (RT).

The mixture was cooled down to RT and Phenoxyethanol (AND) PIROCTONE OLAMINE was added when a temperature of 45° C. was reached. The mixture was stirred 5 minutes and Sodium carbomer was added. The mixture was stirred for 3 minutes. Then the stirring was stopped for 15 minutes. When the temperature of the mixture reached 30° C., the stirring was switched on for another 15 minutes until the cream was homogeneous, glossy and free of lumps. If necessary, the pH was adjusted to 6.70-7.20 with Glydant, Phenonip or Nipaguard PO5, or the pH was adjusted to 6.30-7.00 with Nikkoguard.

### Example 12. Evaluation in Body Lotion Composition

A dilution of trans-2-nonenal (“old people malodor”) at 0.1% in MIP was used as a malodor reference.

A mixture of ethyl acetoacetate and ethanolamine was weighed using a ratio of 90%:10% and applied to the body lotion composition according to Example 11 at a dosage of 0.3%.

Two small wool swatches (11 cm×6 cm, 1.00 g), one for the malodor reference (“sample A”) and one for the body lotion (“sample B”) were evenly sprayed with 0.40 g of water. Using a dropper, 0.25 g of the malodor reference were then evenly distributed at the center of each swatch. The wool swatches were rubbed 10 times and kept for 15 minutes on a hot plate at 32° C. After the 15 minutes, 0.6 g of the body lotion containing the actives (acetoacetate and ethanolamine) was evenly applied on the active sample B swatch.

The swatch was kept for another 15 minutes on the hot plate at 32° C. to let it dry. The 2 wool swatches, the malodor reference sample A and the active sample B, were then placed in separate 20 ml SPME vials and let at room temperature for 2 hours. After 2 hours, the degradation of the trans-2-nonenal as determined by the presence of remaining trans-2-nonenal was measured by GC-MS, from the headspace above the swatch.

The analysis was performed at room temperature with a GC-MS (GC Agilent 5975 B) using a SPME method (1 g sample/Column DB-1 ms, length 30 m×I.D 25 mm×film 0.25 μm/Helium flow 1.2 ml/min, pressure 80.5 Kpa/Split 20/60° C. 2 min, 7° C./min until 250° C.).

The GC-MS analysis was repeated after 24 hours. The results of these analyses are reported in Table 19 below.

TABLE 19

| | Products | |
|---|---|---|
| | Sample A: | Sample B: |
| | Malodor | Active |
| | reference | sample |
| Ingredients | Amount (g) on wool swatches | |
| Trans-2-nonenal dilution at 0.1% in | 0.25 | 0.25 |
| MIP | | |
| Mixture ethyl | — | 0.6 |
| acetoacetate:ethanolamine (90:10) | | |
| at 0.3% in body lotion | | |

| | |
|---|---|
| | Amount (%) of remaining malodor |
| Old people | measured by GC-MS |

| malodor | 2 h | 24 h | 2 h | 24 h |
|---|---|---|---|---|
| Trans-2-nonenal | 100 | 100 | 62.13% | 40.00% |

The measurements summarized in Table 18 show that the mixture of ethyl acetoacetate and ethanolamine (90:10), when applied in a body lotion at 0.3%, significantly reduced the presence of trans-2-nonenal.

The samples were olfactively evaluated out of the jar by an expert panel of 5 people. An evaluation scale between 1-7 was used (1=no malodor//7=very intense malodor). The results of the olfactive analysis are reported on Table 20 below.

TABLE 20

| | Malodor Intensity | Malodor Intensity |
|---|---|---|
| Samples | (1-7) after 2 h | (1-7) after 24 h |
| A: Old people malodor | 7 | 6 |
| reference | | |
| B: Active sample | 3 | 1.5 |

The malodor reduction was also well noticed olfactively (Table 20) with a delta of 4.5 between the old people malodor intensity reference vs. Sample B after 24 hours.

The disclosed subject matter has been described with reference to specific details of particular embodiments thereof. It is not intended that such details be regarded as limitations upon the scope of the disclosed subject matter except insofar as and to the extent that they are included in the accompanying claims.

Therefore, the exemplary embodiments described herein are well adapted to attain the ends and advantages mentioned as well as those that are inherent therein. The particular embodiments disclosed above are illustrative only, as the exemplary embodiments described herein may be modified and practiced in different but equivalent manners apparent to those of ordinary skill in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the exemplary embodiments described herein. The exemplary embodiments described herein illustratively disclosed herein suitably may be practiced in the absence of any element that is not specifically disclosed herein and/or any optional element disclosed herein.

1. A fragrance composition, comprising:
a) an active component comprising at least one 1,3-dicarbonyl compound and at least one basic compound, wherein the amount of the 1,3-dicarbonyl compound is at least 20%, more typically at least 30%, by weight relative to the total weight of the composition; and
b) at least one ingredient selected from the group consisting of a perfumery carrier and a perfumery base, wherein the at least one ingredient is different from the at least one 1,3-dicarbonyl compound.

2. The fragrance composition according to claim 1, wherein the at least one 1,3-dicarbonyl compound is a compound represented by formula (I), 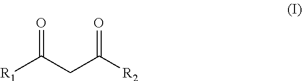
wherein
R₁ and R₂ are each, independently, H, OH, C₁-C₂₀ alkyl, typically C₁-C₁₀ alkyl, more typically, C₁-C₆ alkyl, or C₁-C₂₀ alkoxy, typically C₁-C₁₀ alkoxy, more typically C₁-C₆ alkoxy; or
R₁ and R₂ together form a 5- or 6-membered carbocyclic ring, optionally interrupted by one or more heteroatoms; wherein R₁ and R₂ may optionally comprise one or more substituents selected from the group consisting of halogens, aryl, alkoxy, CN, NO₂, OH, SH, SCH₃, NH₂, COOH, and CONH₂.

3. The fragrance composition according to claim 1 or 2, wherein the at least one 1,3-dicarbonyl compound is ethyl acetoacetate.

4. The fragrance composition according to any one of claims 1-3, wherein the amount of the at least one 1,3-dicarbonyl compound is from 10% to 99%, typically 20% to 99%, more typically 30% to 99%, still more typically 50% to 99%, by weight relative to the total weight of the fragrance composition.

5. The fragrance composition according to any one of claims 1-4, wherein the amount of the basic compound is present in an amount of from 0% to 50%, typically from 0.1% to 40%, more typically 0.5% to 30%, still more typically 1% to 10%, by weight relative to the total weight of the fragrance composition.

6. The fragrance composition according to any one of claims 1-5, wherein the at least one basic compound is a compound represented by formula (II), or a salt thereof, 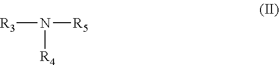
wherein
R₃ is H, aryl, C₁-C₂₀ alkyl, typically C₁-C₁₀ alkyl, more typically, C₁-C₆ alkyl, or CHRₐCOOR_{b}, wherein Rₐ and R_{b} are each, independently, H, C₁-C₂₀ alkyl, typically C₁-C₁₀ alkyl, more typically, C₁-C₆ alkyl;
R₄ and R₅ are each, independently, H, aryl, C₁-C₂₀ alkyl, typically C₁-C₁₀ alkyl, more typically, C₁-C₆ alkyl, or CHRₐCOOR_{b}, wherein Rₐ and R_{b} are each, independently, H, C₁-C₂₀ alkyl, typically C₁-C₁₀ alkyl, more typically, C₁-C₆ alkyl; or
R₄ and R₅ together form a 5- or 6-membered carbocyclic ring, optionally interrupted by one or more heteroatoms;
wherein R₃, R₄, and R₅ may optionally comprise one or more substituents selected from the group consisting of halogens, aryl, alkoxy, CN, NO₂, OH, SH, SCH₃, NH₂, COOH, and CONH₂.

7. The fragrance composition according to any one of claims 1-6, wherein the at least one basic compound has a pKa>9, typically selected from the group consisting of pyrrolidine, ethanolamine, diethylamine, triethylamine, tributylamine, piperidine, alanine, proline, dimethylalkylamines, diethanolalkylamines, and mixtures thereof.

8. The fragrance composition according to any one of claims 1-7, wherein the weight ratio of the at least one 1,3-dicarbonyl compound to the at least one basic compound is from 50:50 to 99:1, typically 80:10 to 95:5, more typically 85:15 to 95:5.

9. A perfumed consumer product comprising the fragrance composition according to any one of claims 1-8.

10. The perfumed consumer product according to claim 9, wherein the perfumery consumer product is a perfume, a fabric care product, a hair care product, a skin care product, a skin-cleansing product, a body deodorant or antiperspirant product, a nail product, or a home care product; typically a hair care product selected from the group consisting of a shampoo, rinse-off conditioner, and leave-on conditioner.

11. The perfumed consumer product according to claim 9 or 10, wherein the perfumery consumer product is a skin care and/or a skin cleansing product selected from the group consisting of a body cream, a face cream, a body-or-face cream, a shower gel, a soap bar, and a body scrub.

12. The perfumed consumer product according to any one of claims 9-11, wherein the amount of the at least one 1,3-dicarbonyl compound is from 0.01% to 4%, typically 0.05% to 2%, more typically 0.1% to 1.5%, by weight relative to the total amount of the perfumed consumer product.

13. The perfumed consumer product according to any one of claims 9-12, wherein the amount of the at least one basic compound is from 0.0001% to 2%, typically 0.0025% to 1%, more typically 0.0025% to 0.5%, still more typically 0.02% to 0.3%, by weight relative to the total amount of the perfumed consumer product.

14. Use of the fragrance composition according to any one of claims 1-8 or perfumed consumer product according to any one of claims 9-13 to prevent, reduce, or ameliorate malodorous aldehydes and ketones from the hair, scalp, skin, or any combination thereof, of a subject, typically a human being, or from any inanimate surface, typically a fabric.

15. The use according to claim 14, wherein the malodorous aldehydes comprise 2-nonenal.
